(19) United States
(12) Patent Application Publication
(10) Pub. No.: US 2011/0250937 A1
(43) Pub. Date: Oct. 13, 2011

(54) Race participant tracking via wireless positioning technology and near real time reporting of location and pertinent race metrics to the participant and optionally to other individuals or publication on the internet

(76) Inventors: Jason R. Hubbard, Brentwood, TN (US); Floyed Jeffries Duncan, JR., Brentwood, TN (US)

(21) Appl. No.: 12/798,680

(22) Filed: Apr. 12, 2010

(51) Int. Cl. A63F 9/24; A61B 5/01; A61B 5/024; A61B 5/08; G01S 19/42; A61B 5/00

(52) U.S. Cl. 463/6; 342/357.25; 600/300; 600/508; 600/529; 600/549

(57) ABSTRACT

Method of tracking race participants via the Global Positioning System, providing near real time feedback to the race participant, and optionally to others individuals or to publication on the web at the user's preference. Feedback to include graphical and tabular presentation of such information as geographic position, race route, current race performance metrics, projection of future milestone and final race performance, projected position at a given time, projected time to a given position, relative performance to historical participants, to personal historical performance and to other members of the current race that are being tracked in aggregate or by demographic or otherwise subdivided

### DEFINITIONS

Wireless Positioning Technology—While specifically referring to the government maintained satellite GPS (Global Positioning System) system currently available, the patent scope should be taken broader to include any service available on a local or global basis through which near real time positions of race participants can be determined for use in the method described herein.

Biometric Data—For the purpose of this concept, biometrics shall include any physical measurements that can be captured by or delivered to the Local Device about the race participant and/or their equipment. Such measurement information might include but not be limited to heart rate, pulse rate, respiratory rate, body temperature and stride/pace in the case of the participant.

Local Device(s)—For the purpose of this concept, the local device is a commercially available smart system comprising at a minimum a microprocessor, display and communications systems. Such systems include PDA devices, cell phones and other such systems specifically including but not limited to brands such as Apple's iPhone and Rim's Blackberry devices. The local device used by the race participant will be GPS enabled or otherwise capable of determining its location.

Local Equipment Data—For the purpose of this concept, local equipment data shall include any physical measurements that can be captured by or delivered to the Local Device about the race participant's equipment. Such measurement information might include but not be limited to operating temperatures, pressures, speeds, accelerometer readings, levels of consumables and the like.

Server—For the purpose of this concept, the server should be consider one or more remote computing, communications and storage devices accessible by the local devices and capable of receiving, storing, manipulating and transmitting data to and from users and observers during a race as well as providing access to data in its storage. The server will provide adequate security to ensure servicing only communications with adequate authority.

Virtual Racer(s)—For the purpose of this concept, a virtual racer is a set of data representing a runner that is not physically in the race it is associated with. This data may represent a runner of notoriety such as last year's winner, or a theoretical racer that might be defined to provide a desired pace. Users and observers can enable virtual racers on their displays to track them relative to actual participants.

### BACKGROUND OF THE INVENTION

This method is pertinent to the realm of racing over a broad geographical area (relative to the resolution of Wireless Positioning Technology) such as cross country or marathon running, sailing races or regattas, biking, road rallies and the like. There exists an unmet opportunity to provide the race participant with valuable feedback thru the interpretation of data collected by Wireless Positioning Technology and ancillary data that is readily available prior to and during the race. Such ancillary data might include information such as Biometric data, Local Equipment Data, the race route, the positions of other participants of the race using the art described herein. Other ancillary data might include historical performance of the participant or other participants potentially of notoriety who have raced the same course. Such data could be utilized either individually or in aggregate.

As is typical of racing enthusiasts, technology is being utilized in increasing innovative manners to monitor personal information and performance during a race and training. Typically however this has been limited to locally collected and communicated data such as heart rate, pedometer derived pace and strides and the local presentation of GPS data. The opportunity to capture and provide more rich data and information based on the interpretation of this data will be appreciated by race participants and the innovative approach to doing so described herein has yet to be demonstrated or protected as intellectual property.

A patent search of prior art in the area of this invention uncovered a limited number of patents of which the methods described herein are distinctive and unique. Specifically differentiation from the most pertinent prior art is described below:

U.S. Pat. No. 5,731,788 teaches of a system as follows:
“A system and method for positioning control and management of racing sailboat positions and velocities includes the strategic placement of global positioning receivers and transmitters at a buoy and committee boat marking the sail race start line, as well as radio and global positioning receivers on the sailboat. Global positioning system (GPS) and radio transmitter units are mounted on a race start buoy and committee boat and another GPS and radio transceiver unit receives GPS signals from positioning satellites and radio signals from the race start buoy and committee boat. The information received by the racing sailboat is processed to determine relative and absolute positions and velocities, and estimated time of arrival (ETA) at the intercept between current sailboat course and the race start line for display in user-friendly race management.”
   The method described above requires the use of GPS technology at three or locations, the start buoy, the committee boat and a racing boat. These three locations are then used to extrapolate the boats current speed vector across a start line formed by the start buoy and the committee boat. The method claimed within, required the use of only one GPS and is therefore unique from the U.S. Pat. No. 5,731,788 scheme.

U.S. Pat. No. 6,744,403 teaches of a system as follows:
“A system uses GPS receivers and other sensors to acquire data about one or more objects at an event. The data acquired by the GPS receivers and the sensors is used to determine various statistics about the objects and/or enhance a video presentation of the objects. In one embodiment, the acquired data is used to determine three dimensional positions of the objects determine the positions of images of the objects in a video and enhance the video accordingly. One exemplar use of the present invention is with a system for tracking automobiles at a race. The system determines statistics about the automobiles and enhances a video presentation of the race.”
   The U.S. Pat. No. 6,744,403 patent above describes primarily of an approach to enhance the display of a video feed of the actual race. This is accomplished in a closed environment prior to production of the enhanced video and does not provide feedback to the participant of the race or directly to observers of the race as taught in the attached patent. In addition, independent claim 1 requires the transmission of differential GPS data, which the proposed patent does not. Independent claims 3 & 4 require the highlighting of objects in a video which the proposed patent does not. Independent claims 5, 16, 21, 27, 43, 46, 70, and 79 require the receiving of video which the proposed patent does not. Independent claim 81 requires the system to determine a position of an image of said moving object in a video which the proposed patent does not. All remaining claims in the 403 patent are dependent on the claims above which the patent herein has been shown to be non-infringing.

U.S. Pat. No. 7,532,977 teaches of a system as follows:
“A portable personal positioner for determining the geographical position of the user without employing the Global Positioning System (GPS) comprising a signal/position display and a traveling speed/distance measuring sensor, wherein the signal/position display is equipped with microprocessor, magneto-resistive sensor and altimeter to measure traveling direction signal along X and Y axis and position signal along Z axis of the use respectively, and to transmit the measured signals to the microprocessor for recording and access, meanwhile the traveling speed/distance measuring sensor is also to measure traveling speed and distance of a user and transmit the measured signals by means of wired or wireless transmission to the microprocessor for recording and access, after data processing from the microprocessor, to obtain the data of traveling speed, traveling distance, geographical position and track of traveling of the user within a time period and display on LCD screen of the signal/position display to determine geographical position without employing the satellite navigation system of GPS; besides, the personal positioner may be further equipped with a cardiac pulse detector for detecting the pulse of heart of a user, and the detected pulse signal is transmitted to the microprocessor of the signal/position display by means of wired or wireless transmission for recording and access to display electrocardiogram or cardiac pulse signal of a user on LCD screen of the signal/position display.”
   The method described above specifically disclaims the use of a GPS device as input to the data collected. While the subsequent use of data obtained otherwise bears some similarity to portions of the method taught herein, the origin of the data not “employing the Global Positioning System” precludes infringing on this patent.

U.S. Pat. No. 7,474,896 teaches of a system as follows:
“A mobile controller unit has a radio positioning system and a two-way communication system and a rover unit also has a radio positioning system and a two-way radio communication system. The controller unit can query the rover unit to send its location data so that the rover unit can be located and if desired, found, such as in the case of a lost child or items. Relative position between the controller and the rover can be displayed on the controller along with an arrow showing where the rover is, as well as how fast it is moving, a track of its movement and other data. The system can also operate in conjunction with a network system that has a PDE and an application server that perform some of the communications and calculation functions.”
   This patent calls for the use of a mobile controller unit, which has two way communications with a rover unit allowing the polling of location information. Our control server, which may be considered analogous to the “mobile controller unit”, is not portable, nor does it have the ability to poll the rover unit. In the method taught herein, the portable unit will routinely and proactively report its location to the server.

U.S. Pat. No. 7,534,206 teaches of a system as follows:
“Navigation-assisted fitness and dieting device A navigation-assisted fitness device broadly comprising a location determining component, a fitness monitoring component and a display. The location determining component determines location data for a user of the device as the user exercises. The fitness monitoring component is coupled with the location determining component and calculates fitness information for the user as the user exercises. The display displays the fitness information and other information and data. The fitness information may include an indication of the intensity level of the user's exercise, the total amount of calories burned by the user during the exercise, the amount of fat calories burned, and the amount of carbohydrate calories burned. The device calculates the ratio of fat calories burned versus carbohydrate calories burned based on the intensity level. As the user increases or decreases the intensity level of an exercise, the device recalculates and displays the ratio or percentage of total calories burned from fat reserves and from carbohydrate reserves.”
   The method described above utilizes location determining component to determine the device that a user is exercising on, it does not utilize this information to determine the position of the user along a race route as taught in the patent proposed herein. While the data displayed on the device described above is similar to portions of that taught herein, this data displayed in a local device can be demonstrated in numerous other examples of prior art such as heart and respiration monitoring watches and the like and is therefore not protect nor infringed by the proposed patent.

U.S. Pat. No. 7,233,795 teaches of a system as follows:
“Location based communications system A location based communications system uses a physical location system (PLS) or relative position system to determine a communication unit's location in two or three dimensions, and is configured for radiotelephonic calls through intermediary communications satellite systems, cellular systems or other mobile systems and land line systems, based on the location G# of the called telephone or on a caller-defined estimated area or space termed a G zone number GZ# surrounding the desired callee. The system is particularly advantageous for mobile communications, and may be interfaced with conventional stationary telephones, cellular phones and other communications devices. Multiple telephone units may be simultaneously contacted with messages in user-defined languages. Emergency features enable the location based communication system to locate and notify the nearest and/or best equipped party for assistance in the event of an emergency, issue warnings to all phones in a specific area of any size, calculate vehicle velocities and control traffic.”
   This system describes the calculation of a velocity of a vehicle, however it differs fundamentally in that the mobile device transmission is utilized as the source of triangulation for determining position via remote receivers where as remote transmitters, satellites, are utilized in the method taught herein to allow location calculation in the remote device.

U.S. Pat. No. 7,158,912 teaches of a system as follows:
“Mobile GPS systems for providing location mapping and/or performance data A location measurement system comprises: a GPS receiver for attachment to a person and for determining earth location of the person; a display for attachment to the person; memory for storing map data; a processor configured to process earth location and the map data to instruct display the person's current location with a map on the display. Other GPS systems for example determine speed with or without map capability or altimeters.”
   This patent teaches the basic technology of a hand held GPS with a tracking memory. The method of the proposed patent utilizes this fundamental GPS data to create unique information and presentation thereof specifically pertaining to a race participant and to interested observers of a race participant(s).

U.S. Pat. No. 6,662,016 teaches of a system as follows:

“Providing graphical location information for mobile resources using a data-enabled network The inventive system relates to applications for providing graphical display information regarding mobile resources. In the illustrated embodiment, the system (100) provides graphical location information regarding one or more mobile resources (110, 120 and/or 130). The graphical location information is provided via a display (147) associated with a client platform (140). The display includes mapping information and mobile resource marker information transmitted from a server (170) to the client platform (140) via the Internet (180). The mapping information and marker information are provided in separate message sets thereby providing certain processing advantages.”

The U.S. Pat. No. 6,662,016 patent's independent claim #1 requires that the determination of location of the mobile device “is based on the location of said mobile resource in relation to at least one fixed ground-based wireless network structure having a known geographic location”. The application for protection herein has no such requirement. All other claims of this patent are dependent on claim 1 and therefore also excluded.

Classification of the invention herein is anticipated as follows:
CLASS 342 Communications: directive radio wave systems and devices (e.g., radar, radio navigation
   357.07 . . . Tracking or monitoring
   357.1 . . . Combined with telecommunications:
CLASS 701, DATA PROCESSING: VEHICLES, NAVIGATION, AND RELATIVE LOCATION
   201. Determination of travel data based on the start point and destination point:
   204 Determination of E.T.A.: This subclass is indented under subclass 200. Navigation system wherein the data processing system or calculating computer functions to determine the time of arrival at a destination.
   213 Using Global Positioning System (GPS):

### BRIEF SUMMARY OF THE INVENTION

This invention describes a method wherein the participant in a race or practice/training for a race is tracked via a local smart device providing a plurality of metrics potentially including but not limited to:
Location, speed and direction based on a Wireless Positioning Technology
Biometric data
Local Equipment Data

The method further anticipates the optional entry or download of previously entered planned race locations and routes that can be used for display purposes and to predict/extrapolate to future events such as time and elapsed time for arrival at an intermediate point or at the end of the race.

Also envisioned is the ability to compare the metrics mentioned above to benchmark values that may be from the participants own history, custom developed benchmarks, historical benchmarks from other race participants or any combination of the aforementioned.

Also envisioned is the ability of the smart device to communicate bi-directionally with a central server where information can be archived, consolidated and viewed via the server over the internet.

Further envisioned in the ability for the aforementioned server to communicate selected and authorized portions of the information it received to the race participant or other remote users via wireless communication to their Local Devices providing display and/or query capabilities.

Also envisioned is a mode whereby a third party can, with proper credentials, view the progress and metrics of one or more race participants simultaneously either via the web or via a Local Device.

Further envisioned is the ability of the system to accept and respond to inquiries as to the projected location at a given or incremental time, or conversely to project the time or incremental time of arrival to a given location.

It is anticipated that thru a customizable screen display that the user can view a plurality of metrics in near real time during the race using their Local Device. The metrics might include but are not limit to such measures as the following:
Current speed
Current pace/stride (in the case of a runner)
Speed average over the past specified period
Average speed since beginning of race
Current speed as a percent of an average speed mentioned above
Heart rate
Respiration rate
Body temperature
Other biometric metrics as might be externally measured and accessible by the Local Device
Ambient temperature
Calories burn rate and total calories burned over a specified period
Local Equipment data
Current position in race relative to a reference group such as:
   Other participants currently being tracked
   Historical performance of the participant, others, a theoretical performance or a consolidation of multiple of the aforementioned in this or a similar race
   A subset of the above based on age, gender, elite participants or other forms of segregation

It is also envisioned that graphical representation of the information available will be offered on the Local Device. These presentations might include but are not limit to the following:
A graphical street map view or satellite image view of the course or a part thereof
The above view with the route superimposed
The same views with the location of the participant and/or others and/or virtual racers superimposed

Information and graphics described in the previous two sections may also be provided, with proper credentials, to observers of the race and may be done so simultaneously for multiple participants.

The Server is envisioned to have the capability of archiving, summarizing, trending and providing query capability of the data communicated to it. This will allow such analysis as training progress, comparisons to benchmarks, recall of previous performance or ad hoc queries to be performed as may be desired by the user. Proper credentials would be required to access said data.

### DESCRIPTION OF THE DRAWINGS

FIG. 1 describes the overall concept in a block diagram. 1.1 Represents a Local Device being located with a race participant. This device will be in routine reception of GPS satellites 1.2 or other wireless positioning technology capable of determining its location. The local device will obtain via the wireless positioning technology and/or local equipment data sensors (1.8) and calculations such data as position, speed, pace, temperature, heartbeat etc. This data will then optionally be displayed via characters or graphics on the local device display. In addition the data will periodically be communicated via a wireless link 1.6, such as the cell data network, optionally thru a global network 1.7 and to a remote server 1.3. The server will subsequently provide access to other users who have proper credentials the ability to access the data available thru the internet or other data network 1.7. This will be accomplished by other users (observers) displaying similar graphics or textual information as available to the race participant either on their Local devices 1.4 or via the web on a personal computer client 1.5

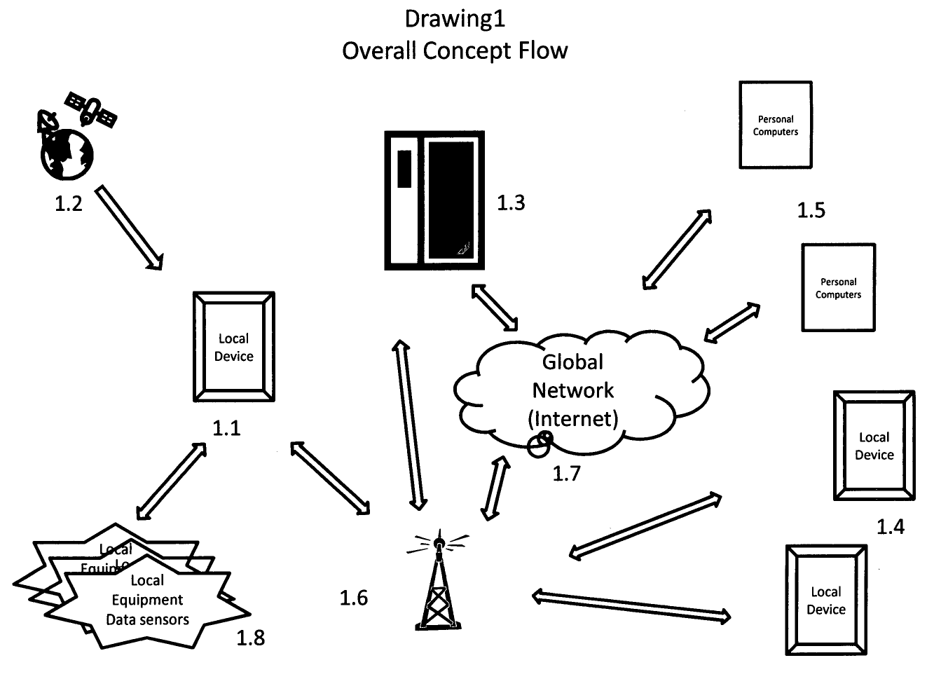

FIG. 2 describes the initial options of the preferred embodiment of the Local Device and is reference in the description thereof.

### DETAILED DESCRIPTION OF THE INVENTION

In the preferred embodiment of this concept an iPhone application will be developed and made available via online download that will implement the envisioned functionality. The functionality to be enabled for a runner, specifically a Marathon runner is envisioned as the first application to be produced and is described below as a preferred embodiment.

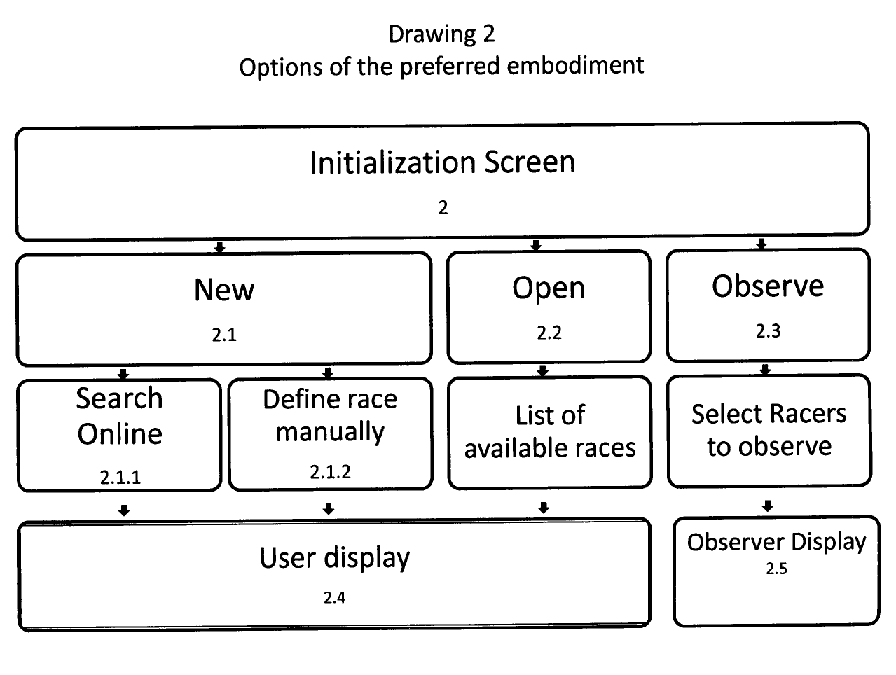

After downloading, installing and activating the application the user will be presented with 3 options for usage as shown in FIG. 2 (2)

The first option; “NEW” referenced as 2.1 allows the user to add a route not previously stored in the device. This selection in turn allows the user two subsequent options shown as 2.1.1; “Search Online” and 2.1.2 “Define race manually”. If “Search Online” 2.1.1 is selected the user will be provided assistance via a configurable search screen to search available routes for download and the ability to purchase and download the selected route. If the “Define race manually” option 2.1.2 is selected, the user is provided a graphical map interface to locate the start point, intermediate turns or via points and an end or finish point which would define a custom route to be run. That route would then be saved to the device and in the users account on the server. In either case, the user would then be presented a graphical map of the race selected with the current GPS location overlaid and a user defined selection of the available metrics shown designated as 2.4 and described below.

The second option shown in FIG. 2 is designated 2.2 “Open”. Selecting this option will allow the user to view a list of races previously downloaded or manually defined. The user will then select the race desired and would then be presented a graphical map of the race selected with the current GPS location overlaid and a user defined selection of the available metrics shown designated as 2.4
The 2.4 display would afford the user an ability to pan, zoom and auto track on the map. Options would be made available to modify the metrics being shown and perform other administrative tasks such as enabling/disabling observers, allowing public observation, setting passwords, defining reporting frequencies. Additional options would be provided for enabling external devices for ancillary data collection such as heart/respiration monitors or temperature/humidity monitors. The racer will also be able to enable observation of other race participants on his local display. The ability to start and or stop the race either manually or automatically based on GPS location will be provided.

The Third option from the main screen designated as 2.3 will be utilized by observers, allowing the same client software to be utilized by both the race participate and race observers. When the 2.3 option is selected the observer would be required to enter the ID (typically an email address) of the race participant(s) they wish to monitor, their own ID (typically and email address) and an associated security code provided them by the race participant if required. Once a race is identified by the first participant, they observer will also be able to select other participants from that race who have made their data feed public or who have provided permission. The observer would then be presented a graphical map of the race selected with the current GPS location overlaid and a user defined selection of the available metrics shown designated as 2.5. Screen 2.5 will be similar to 2.4, but not present some of the race participant specific options such as enabling external devices for ancillary data collection, enabling observers etc.

In both 2.4 and 2.5 screens a “Virtual racer” can also be enabled for observation if available from the server. This virtual racer would represent the performance of an historical or theoretical race participant and may be the previous year's winner, the participants previous year performance or a theoretical performance to pace the runner or similar. The virtual racer would be overlaid on the map in a method similar to actual race participants.

Periodically, at a frequency defined by the race participant, the current location and biometric data and local equipment data will be communicated wirelessly to the server. The server will archive this data for later retrieval with proper credentials and will provide access to the data in near real time to Observers. The server will also host available maps of planned race routes and virtual participants historical performance data which will be accessed on demand as described above. The server will have the ability to maintain multiple races run on the same route at different times, by different runners/virtual racers and will provide graphical and tabular comparisons of the same for display and analysis.

1) We claim,
a software solution running on a portable device accompanying a race participant such as a cell phones, PDA, BlackBerry™, iPhone™ or the like, implementing a method to track or download, calculate as necessary and display one or more of the following metrics:
Participant location via GPS or other wireless positioning technology,
Map and satellite representations of the location of the race
distance since beginning of event,
elapsed time since beginning of event,
average pace since beginning of event,
pace in a recent time period,
pace over a recent distance,
indication of change of pace over time and/or distance,
distance remaining to finish,
estimated time to arrival at milestone locations,
estimated time of arrival at event end,
estimated elapsed time to arrival at milestone locations,
estimated elapsed time at event end,
estimated calories burned,
heart rate,
Change in heart rate over time and/or distance,
Respiration rate,
Body temperature,
Other biometric metrics as might be externally measured and accessible by the local device,
Local equipment data,
Ambient temperature, and
Ambient humidity.

2) The method in claim 1 with optional entry or selection of configuration information of the event and participant prior to the event beginning via the portable device or via the web including one or more of the following:
event starting point,
event ending point,
route to be followed during the event,
event distance,
customized milestone locations,
participant identifying information,
participant age group,
participant gender,
participant race ID info,
setup of observers permissions
participant RFID info such as Champion Chip I.D.®, or
Benchmark historical or custom race performance.

3) The method of claim 2 with the optional overlay of a graphical street view or satellite view display with various information including one or more of the following:
starting point
ending point
current racer location
current location of other racers or virtual racers
route to be followed during the event
one or more of the metrics in the step above

4) The method of claim 3 with the optional communication of participant data to a central server where one or more of the follow functions will occur;
Recording of information for archiving and subsequent retrieval
Access to a graphical or tabular representation of the data via the web view to authorized users
Access to estimated elapsed and clock time to a specified location
Estimation of location at a specified time or elapsed time from current

5) The method in claim 4 wherein the data reported to a central server is consolidated with that of other race participants which are utilizing the program and optionally with other historical, theoretical or user defined profiles and calculating and presenting one or more of the following:
Graphical location of individuals or average location of other participants
Percentage position relative to other participants
Percentage position relative to other participant in gender group
Percentage position relative to other participant in age and gender group
Percentage position relative to other participants adjusted for age and gender
Percentage position relative to benchmark performances selected
Percentage position relative to elite performing groups

6) The method described in claim 4 where access to available information and presentation formats are further communicated to a remote viewer who may not be in the race, using a PDA, cell phone or other such portable device or via a web browser.

7) The method described in claim 5 where access to available information and presentation formats are further communicated to a remote viewer who may not be in the race, using a PDA, cell phone or other such portable device or via a web browser.
